(19) United States
(12) Patent Application Publication
(10) Pub. No.: US 2008/0090243 A1
(43) Pub. Date: Apr. 17, 2008

(54) PSEUDO-TISSUES AND USES THEREOF

(75) Inventor: Matthew Baker, Maidstone (GB)

Correspondence Address: INVITROGEN CORPORATION;C/O INTELLEVATE, P.O. BOX 52050, MINNEAPOLIS, MN, 55402, US

(73) Assignee: INVITROGEN CORPORATION, Carlsbad, CA (US)

(21) Appl. No.: 11/873,313

(22) Filed: Oct. 16, 2007

Related U.S. Application Data
(63) Continuation of application No. 10/443,969, filed on May 23, 2003.
(60) Provisional application No. 60/382,389, filed on May 23, 2002.

(51) Int. Cl. C12N 5/00; C12Q 1/68; G01N 1/28

(52) U.S. Cl. 435/6; 435/325; 435/40.52

(57) ABSTRACT

The invention provides compositions and methods useful in the analysis of tissues and cells. More specifically, the invention provides compositions, referred to as “pseudo-tissue samples,” which comprise aggregated fixed cells embedded in an embedding medium. The invention also provides histological specimens comprising pseudo-tissues, as well as histological specimen-substrate compositions, such as microscope slides upon which pseudo-tissues and histological specimens prepared therefrom are placed. The histological specimen-substrate compositions may comprise arrays (or microarrays) of pseudo-tissues and/or histological specimens. The invention also provides methods for preparing pseudo-tissues, histological specimens, and histological specimen-substrate compositions containing the same. Also provided is a method for analyzing a tissue sample by comparing the subcellular features of a reference histological specimen to those found in a candidate histological specimen, such that one or more subcellular features shared by both the candidate and reference histological specimens may be identified. The subcellular features that may be identified include, e.g., DNA, RNA, proteins, enzymes and carbohydrates.

### BACKGROUND OF THE INVENTION

1. Field of the Invention

The present invention relates generally to compositions and methods useful in the analysis of tissues and cells. More specifically, the invention relates to pseudo-tissue samples, histological specimens and arrays comprising pseudo-tissue samples, and methods of preparing the same. The invention also relates to the histological analysis and/or comparison of subcellular features within tissue samples and within clonal populations of cells. The subcellular features that are analyzed in the tissue samples and cells include, for example, cellular morphology, nucleic acids, proteins, carbohydrates, and enzymatic activity.

2. Background Art

The diagnosis and prognosis of disease conditions often require that a tissue sample be removed from a patient and processed for histological examination. The characteristics of a tissue sample often provide important information regarding the health of an individual. In addition to diagnostic applications, histological analysis of tissue samples can also be used in conjunction with high-throughput screening methods to identify particular subcellular features in an array of tissue samples.

Biological tissues are commonly analyzed by removing a tissue sample from a living organism, fixing the sample, embedding the fixed tissue in a material such as paraffin, and slicing the embedded fixed tissues into very thin sections. This procedure produces a histological specimen of very high quality.

Alternatively, the specimen can simply be frozen, cut, and mounted on a slide. This “frozen section” procedure has the advantage of enabling a rapid histological diagnosis to be made from the specimen, and it is frequently employed in situations where a diagnosis is necessary while a patient is on an operating table. There are certain disadvantages, however, associated with the use of frozen sections. For example, the prepared slide does not possess the uniform quality of morphology of samples prepared by other methods.

The histological specimens prepared according to either the paraffin-embedding method or the frozen section method can be analyzed in a variety of ways, such as staining the sample to identify nucleic acids, or probing the sample with detectably-labeled antibodies.

Biological analysis using thin sections of embedded fixed tissue, is frequently used in the diagnosis and prognosis of diseases and conditions such as cancer. For instance, in the case of suspected cancerous tissue, a biopsy may be performed to determine whether the tissue is cancerous. In a “core biopsy,” for instance, a core or fragment of tissue is removed from the patient and a histological specimen is prepared from the tissue. The specimen is analyzed microscopically to determine whether the tissue exhibits the hallmarks of neoplasia or cancer.

Fixed, embedded tissue samples can provide a source of archival tissues that can be analyzed several years after the tissue has been removed from the patient. In one study, it was concluded that archival tissue samples up to 68 years old remain suitable for immunohistochemical analysis. (Camp, L., et al., Laboratory Investigation 80:1943-1949 (2000)). Thus, archival specimens can be used to obtain long-term follow-up data regarding a particular tissue.

Multiple tissue samples can be analyzed simultaneously using tissue microarrays. Tissue microarrays can be constructed by combining hundreds of cylindrical tissue samples in a single paraffin block. (Kononen, J, et al., Nature Medicine 4:767-768 (1998)). Each cylindrical sample generally has a diameter of approximately 0.6 mm in diameter. (Schraml, P, et al., Clinical Cancer Res. 5:1966-1975 (1999)). Thin sections from the tissue microarray blocks can be analyzed using a variety of techniques, including DNA and RNA in situ hybridization and immunohistochemistry. (Bubendorf, L, et al., Cancer Res. 59:803-806 (1999)). Immunohistochemical markers or labels are often used in histology for identifying certain characteristics of a cell, for example, whether the cell is undergoing mitosis or expression of a certain antigen.

In contrast to traditional histological techniques, which require the processing and staining of hundreds of slides, microarray technology enables the study of an entire cohort of cases by analyzing just one (or a few) master slide(s). Microarray analysis has the added advantage that all specimens are processed at one time using identical conditions. Furthermore, it markedly reduces the amount of tissue required for a particular study, thus preserving ample remaining tissue for other research or diagnostic needs. (Camp, L., et al., Laboratory Investigation 80:1943-1949 (2000)). In the context of the analysis of cancerous tissue, tissue microarrays facilitate the standardized analysis of multiple genes in the same tumors using the same methods, probes, and interpretation criteria. (Bubendorf, L, et al., Cancer Res. 59:803-806 (1999)).

The analysis of tissue samples can be integrated with the methodology of molecular profiling. In molecular profiling, global gene expression patterns are measured, and the individual genes and collections of genes that mediate particular aspects of cellular physiology are measured. (Emmert-Buck, M. R., et al., J. Molec. Diagnostics 2:60-66 (2000)). In humans, for example, molecular profiling may be used to advance the understanding and treatment of diseases. Measurement of expression patterns of normal and affected cell populations have the potential of identifying specific sets of genes that are abnormally regulated. Moreover, the availability of full-length mRNA coding sequences will allow prediction of function based on computer modeling algorithms, promoting a more fundamental understanding of the disease process as well as new diagnostic and therapeutic targets for clinical intervention. (Emmert-Buck, M. R., et al., J. Molec. Diagnostics 2:60-66 (2000)).

Generally speaking, tissue analysis is accomplished by identifying and characterizing certain subcellular features within a tissue sample being analyzed. The subcellular features that are identified during tissue analysis provide information regarding the status of the tissue sample and ultimately provide insight regarding the health of the patient from which the tissue sample is obtained. Important subcellular features for histological analysis include cell morphology, cytoskeletal architecture, proteins, nucleic acids, carbohydrates, and enzymatic activity.

DNA probes are useful for identifying specific nucleic acids within a cell or tissue sample. For example, fluorescent in situ hybridization (FISH) using labeled DNA probes is useful in detecting particular regions on a chromosome. According to the FISH technique, a DNA probe is labeled with fluorescent molecules (direct method) or non fluorescent molecules which are then detected by fluorescent antibodies (indirect method). The probes bind to a specific region or regions on the target chromosome. The chromosomes are then stained using a contrasting color, and the cells are viewed using a fluorescence microscope. FISH can be used, for instance, in the detection of gene amplifications relating to cancer. (Bubendorf, L., et al., Cancer Res. 59:803-806 (1999)). Combinatorial labeling of DNA probes using two or more different reporters has been described, wherein the number of distinguishable targets relative to the number of available fluorophore detectors is markedly increased. (Ried, T., et al., Proc. Natl. Acad. Sci., 89:1388-1392 (1992)).

In addition to nucleic acids, various proteins or polypeptides can also serve as important diagnostic subcellular features that can be detected within a tissue sample. Proteins are most commonly detected using labeled antibodies. In many cases, the antibodies are labeled fluorescently. Fluorescent antibody techniques involve a variety of methods including direct fluorescent, indirect fluorescent, mixed antiglobulin, and sandwich techniques. The direct fluorescent staining reaction involves a process, wherein the fluorescent-labeled probe, such as an antibody is specific for the polypeptide (i.e., antigen) of interest. Another direct technique involves a “sandwich” reaction used to identify antibody rather than antigen in tissue samples. Antigen is added to tissue and is bound by specific antibody present in the cell. A labeled antibody specific to the antigen is added and reacts with the antigen, which has been fixed to the antibody in the cell.

Indirect fluorescent staining reactions may involve a multiple-step process, wherein the first step uses an unlabeled antibody (i.e., the primary antibody) that is specific for an antigen. Subsequent steps utilize a fluorescent-labeled antibody of another species (i.e., a secondary or tertiary antibody) that binds to the unlabeled primary antibody. Another indirect method involves a mixed antiglobulin reaction, wherein antigens present on the primary antibody are used to react to binding sites on the secondary antibody. The immunoglobulin antigens are present on the cell and the anti-immunoglobulin antibody is used to bind labeled immunoglobulin to the cell surface immunoglobulin.

The analysis of subcellular features within a tissue sample, including nucleotides, polypeptides, or other subcellular features, can greatly aid in the diagnosis of diseases and conditions. For example, in the diagnosis of many cancers, an important subcellular feature that can be detected in a tissue sample is the overexpression of known oncogenes. An increase in expression of estrogen receptor, progesterone receptor and the Her2/neu oncogene, for instance, have been shown to be associated with invasive breast carcinoma. (Camp, L., et al., Laboratory Investigation 80:1943-1949 (2000)).

In addition, the analysis of subcellular features within a tissue sample can be useful in identifying cells undergoing programmed cell death, also known as apoptosis. Apoptosis is characterized by a set of morphological and biochemical changes that reflect an active cell suicide. Subcellular features indicative of apoptosis include cell shrinkage, nuclear chromatin condensation and margination, and DNA fragmentation. In addition, biochemical events such as the externalization of phosphatidyl serine and the activation of aspartate-specific cysteine proteases may serve as important apoptosis-related subcellular features. For instance, proteases within the cysteine aspartic acid protease family, also known as the ICE/CED-3 family, are critical for effecting the process of apoptosis. These enzymes are cysteine proteases and exhibit substrate specificity for cleavage after an aspartic acid residue. Due to these characteristics, these enzymes are now referred to by the above term “cysteine aspartic acid proteases” or “caspases.” During the apoptotic process, caspase activity is generated in cells. (Fritz, et al., U.S. Pat. No. 6,270,980 (2001)).

Thus, there are many subcellular characteristics that are indicative of the medically relevant process of apoptosis (or lack thereof). The identification within a tissue sample of such subcellular features can provide important clinical information regarding a patient.

As the above discussion illustrates, the histological tissue analysis of subcellular features is an extremely useful biomedical diagnostic tool. The analysis of biological tissue samples is complicated, however, due to the heterogeneous composition of most tissues. Histological specimens include many complex cellular arrangements and architectures. The cells within a specimen often overlap with one another, and the cell architectures may be presented in various orientations due to the method of slicing used to prepare the specimen.

The problems associated with tissue analysis are particularly pronounced in the context of analyzing specific proteins or nucleic acids in tumor tissue. In particular, studies to quantitatively or qualitatively assess proteins or nucleic acid expression in human tumor cells are compromised by the diverse cell populations present in bulk tumor specimens. Histological specimens of invasive tumor typically show a number of cell types including tumor cells, stromal cells, endothelial cells, normal epithelial cells and inflammatory cells. Moreover, the analysis of cellular tissue material extracted from a patient is limited because the extraction reflects only the average content of disease associated markers. (Bonner et al., U.S. Pat. No. 6,251,516 (2001)).

A region of tumor tissue subject to biopsy and diagnosis as small as 1.0 mm can contain normal epithelium, pre-invasive stages of carcinoma, in-situ carcinoma, invasive carcinoma, and inflammatory areas. Consequently, routine scraping and cutting methods will gather all of these types of cells, and hence, loss of an allele will be masked by presence of a normal copy of the allele in the contaminating non-malignant cells. Existing methods for cutting away or masking a portion of tissue do not have the needed resolution. Hence the analysis of genetic results by those previous methods are always plagued by contaminating alleles from normal cells, undesired cells or vascular cells. (Bonner et al., U.S. Pat. No. 6,251,516 (2001)).

The difficulty in analyzing histological specimens due to the heterogeneous composition of tissue is recognized in the art. In U.S. Pat. No. 6,251,516, for example, the problem is addressed by identifying and extracting specific cells from a cellular tissue sample. The extraction of individual cells, however, can be very time consuming and may require the use of specialized equipment such as lasers and various other cell manipulation devices. (See U.S. Pat. No. 6,251,516 (2001)). Moreover, in many instances, it will be desirable to keep the tissue sample intact for its analysis; for example, when important information is provided by the characteristics of surrounding tissue matter in a sample, it would be counterproductive to isolate and analyze the individual cells.

Although the analysis of intact tissue samples is relatively quick, inexpensive, and provides useful diagnostic information about a patient, there are, nonetheless, many advantages associated with the analysis of individual clonal populations of cells. The difficulties associated with biological tissue analysis are encountered to a much lesser extent when analyzing individual clonal populations of cells such as cultured mammalian cells. Such cells are genetically identical, thus, the problems attributed to the heterogenous composition of tissue samples does not exist in clonal population analysis.

Even finer analysis of subcellular features can potentially be achieved by using hybrid cells. A hybrid cell is a single cell obtained by fusing two or more cells together. One of the cells used in creating the hybrid cell is sometimes referred to as the donor cell if it contributes only a portion of its nucleic acid content. For example, a hybrid cell may contain either a single exogenous chromosome, or a fragment of an exogenous chromosome from the donor cell. (Cox, D. R., et al., Science 250:245-250 (1990)). The exogenous chromosome (or portion thereof) is usually derived from a human donor cell. Thus, the subcellular features observed in a hybrid cell (containing only one or a portion of a chromosome from a donor cell) would be attributed specifically to the particular chromosome or chromosome fragment found within the hybrid cell.

The analysis of clonal populations of cells, such as mammalian tissue culture cells and hybrid cells, does not carry with it certain difficulties associated with whole tissue sample analysis. More specifically, problems caused by the heterogeneous character of tissue samples and the variations involved with tissue sample preparation are not encountered when analyzing cultured clonal cell populations (or hybrids). However, the information derived from analyzing subcellular features in cultured cells is usually of less diagnostic utility than the analysis of such features in tissue samples. Moreover, using current methods for analyzing subcellular structures in cultured cells, it is extremely cumbersome to analyze cell populations in ordered arrays. That is, the subcellular features of cultured clonal populations of cells are typically analyzed by growing the cells directly on a microscope slide, fixing the cells on the slide, and then processing the cells to identify subcellular features such as nucleotide sequences or polypeptides. Because clonal populations of cells are typically processed in this manner, i.e., each slide carrying a single population of cells, it is extremely difficult and time consuming to analyze a multitude of cell populations at once, or to perform side-by-side analyses between a tissue sample and a clonal population of cells. Thus, in order to enhance and simplify the analysis of tissues and cells, it is extremely useful to combine the advantages of analyzing subcellular features of individual clonal cell populations with the diagnostic utility associated with histological analysis on complex biological tissue samples.

### BRIEF SUMMARY OF THE INVENTION

The present invention overcomes disadvantages of the prior art by providing compositions and methods useful in the analysis of tissues and cells. The invention combines the informational and diagnostic utility of traditional histological tissue sample analysis with the versatility and fine molecular detail associated with clonal cell population analysis. The compositions and methods of the invention may be used to help correlate the phenotype of a particular candidate specimen with the genotype of a reference specimen.

The compositions and methods of the invention may be used in processes for identifying particular tissue samples or physiological states of tissue samples. Identification of particular physiological states may then be used, for example, to identify pathological conditions (e.g., cancers, warts, diseases and/or afflictions resulting from increased or decreased apoptosis, etc.). As one skilled in the art would recognize and as discussed elsewhere herein, when tissue samples and cells are analyzed and/or identified, for example, reference can be made to features of the macrocellular character of the tissues or of the individual cells. In either instance, the tissue or cells in the sample are analyzed and/or identified, as this phrase is used herein.

In one aspect of the invention, pseudo-tissue samples are provided. In the context of the present invention, a “pseudo-tissue sample” is a composition comprising an embedding medium and a clonal population of cells. The clonal population of cells in the pseudo-tissue sample comprise fixed cells that have been collected and/or formed into an aggregation and are embedded within said embedding medium.

In another aspect of the invention, histological specimens obtained from pseudo-tissue samples are provided. The histological specimens according to this aspect of the invention may comprise an entire pseudo-tissue sample or a portion thereof, for example, a thin section or core of a pseudo-tissue sample. In any event, the histological specimens obtained from pseudo-tissue samples will be in a form that is suitable for microscopic or other histological analysis known in the art.

In another aspect of the invention, histological specimen-substrate compositions are provided. The histological specimen-substrate compositions according to this aspect of the invention comprise a planar or substantially planar substrate (such as a microscope slide), and one or more histological specimens comprising pseudo-tissue samples, as described supra. The histological specimen-substrate compositions will preferably be constructed such that the histological specimens are in contact with only one side of the substrate. The substrates may contain only one histological specimen, or alternatively, may contain a plurality (for example, from about 2 to about 1000) of histological specimens. When more than one histological specimen is included in the histological specimen-substrate composition, the histological specimens may be arranged on the substrate in an ordered two-dimensional array. In certain embodiments, the arrangement of the histological specimens on the substrate will facilitate the automated analysis of the specimens.

The histological specimen-substrate compositions of the invention may contain, in addition to histological specimens comprising pseudo-tissue samples, standard histological specimens comprising biological tissue samples (i.e., non-pseudo-tissue samples).

The invention also provides methods for preparing the above-described pseudo-tissue samples, histological specimens, histological specimen-substrate compositions, and arrays. For example, methods are provided for preparing a pseudo-tissue sample comprising: (a) obtaining a clonal population of cells, (b) fixing the cells of said clonal population of cells, (c) collecting or forming the fixed cells into an aggregation, and (d) embedding said aggregation of cells in an embedding medium; wherein said embedded aggregation of fixed cells comprises a pseudo-tissue sample.

Methods are also provided for preparing a histological specimen, said methods comprising: (a) obtaining a pseudo-tissue sample; and (b) processing said pseudo-tissue sample so that it is suitable for microscopic analysis. The processing of the pseudo-tissue sample may involve removing a portion of the pseudo-tissue sample. For example, a histological specimen may be obtained according to the present invention by removing from a pseudo-tissue sample a thin section or a core. Methods are also provided wherein a plurality histological specimens, each obtained from a separate pseudo-tissue sample, are placed in an array on a planar or substantially planar substrate (such as a microscope slide).

In another aspect of the invention, methods are provided for analyzing a tissue sample. The methods according to this aspect of the invention comprise: (a) obtaining one or more reference histological specimens, each reference histological specimen comprising pseudo-tissue sample or sub-portion thereof (e.g., thin section, core, or other preparation); (b) obtaining one or more candidate histological specimens, each candidate histological specimen comprising a tissue sample to be analyzed; and (c) detecting one or more subcellular features common to said reference and candidate histological specimens. The candidate histological specimens may be any histological specimen (i.e., pseudo-tissue samples or, preferably, non-pseudo-tissue samples) obtained by any histological preparation method known to those skilled in the art. For example, the candidate histological specimens may be thin sections of fixed biological tissues that have been embedded in paraffin or other embedding medium known to those skilled in the art. According to this aspect of the invention, the tissue samples to be analyzed can be fixed with any fixative known to those skilled in the art. Besides embedded fixed tissue samples, the candidate histological specimens of the invention may alternatively be derived from frozen tissue, or they may be smear preparations.

In another aspect of the invention, methods are provided for identifying a tissue sample that possesses a pre-selected subcellular feature. The methods of this aspect of the invention comprise: (a) obtaining one or more reference histological specimens, each reference histological specimen comprising a pseudo-tissue sample or sub-portion thereof (e.g., thin section, core, or other preparation); (b) obtaining a plurality of candidate histological specimens, each candidate histological specimen comprising a distinct tissue sample to be analyzed for the presence of said pre-selected subcellular feature; and (c) detecting said pre-selected subcellular feature in said reference and candidate histological specimens.

In another aspect of the invention, kits are provided for preparing a pseudo-tissue sample. Also provided are kits for preparing a histological specimen comprising a pseudo tissue sample, or an array of histological samples, each histological sample within the array comprising a pseudo-tissue sample. In one embodiment, kits of the invention comprise: (a) one or more clonal population of cells; (b) one or more solutions comprising a fixative agent; and (c) one or more embedding media; wherein the kit components together facilitate the preparation of a pseudo-tissue sample.

Kits of the invention may additionally comprise one or more of the following: one or more instrument for obtaining a thin section or core from a pseudo-tissue sample (for example, a microtome or razor blade); one or more substrate upon which histological specimens may be placed for microscopic analysis; one or more reagent that facilitates the detection of one or more subcellular features; water; one or more buffer; one or more containers for storing or transporting kit components; computer software that facilitates the automated analysis of an array of histological specimens; or user instructions for preparing a pseudo-tissue sample, a histological specimen, or an array of pseudo tissue samples (e.g., one or more pseudo-tissue sample arrays of the invention).

### DETAILED DESCRIPTION OF THE INVENTION

The present invention is directed to methods and compositions useful in the analysis of tissues and cells. More specifically, the invention provides pseudo-tissue samples, histological specimens comprising pseudo-tissue samples, histological specimen-substrate compositions comprising one or more of such histological specimens, and arrays of pseudo tissue samples. The invention also provides methods for the preparation of the pseudo-tissue samples, histological specimens, and histological specimen-substrate compositions of the invention, including the preparation of arrays comprising pseudo-tissue samples. Also provided are methods for analyzing tissue samples comprising comparing the subcellular features within a candidate histological specimen to the subcellular features within a reference histological specimen. Additionally, the present invention provides kits for preparing the compositions of the invention, as well as kits for practicing the methods of the invention.

Pseudo-Tissue Samples

In one aspect of the invention, pseudo-tissue samples are provided. As used herein, the term “pseudo-tissue sample” is intended to mean a composition comprising an embedding medium and a population of cells that have been fixed, formed and/or collected into an aggregation, and embedded within said embedding medium. The term “pseudo-tissue sample” is also intended to encompass sub-portions, fragments or pieces of pseudo-tissue samples. For example “histological specimens obtained from pseudo-tissue samples,” as described in more detail elsewhere herein are encompassed within the definition of pseudo-tissue samples.

The present invention also provides methods for preparing pseudo-tissue samples. In an exemplary embodiment, methods are provided comprising: (a) obtaining one or more clonal populations of cells, (b) fixing the cells of said one or more clonal populations of cells, (c) collecting or forming the fixed cells into an aggregation, and (d) embedding said aggregation of cells in an embedding medium; wherein said embedded aggregation of fixed cells comprises said pseudo-tissue sample. The invention further includes compositions prepared by such methods, as well as kits for performing these methods.

Pseudo-tissue samples of the invention may comprise a single clonal population of cells (e.g., an embedded aggregation of fixed, clonally-identical cells). In particular embodiments, however, multiple (e.g., 1, 2, 3, 4, 5, 6, 7, 8, 9, 10, 15, 20, 30, 40, 50, 70, 90, 100, etc.) clonal populations of cells may be included within a pseudo-tissue sample. Pseudo-tissue samples comprising multiple clonal populations of cells can be prepared by mixing or otherwise combining two or more fixed clonal populations of cells, collecting or forming the mixed populations of fixed cells into an aggregation, and embedding the aggregation of cells in an embedding medium. Alternatively, two or more clonal populations of unfixed cells may be mixed together, and then fixed, prior to collecting/forming the fixed cells into an aggregation and embedding the aggregation of cells in an embedding medium to form a pseudo-tissue sample.

As used herein, the term “clonal population of cells” is intended to mean one or more cells that share a substantially identical genotype. As appreciated by those skilled in the art, a clonal population of cells may include a small percentage of non-genotypically identical cells that arose from mutation or through minor cross-contamination.

The cells of a clonal population of cells within a pseudo-tissue sample can be any type of cell. For example, the cells may be prokaryotic (bacterial, including members of the genera Escherichia, Serratia, Salmonella, Staphylococcus, Streptococcus, Clostridium, Chlamydia, Neisseria, Treponema, Mycoplasma, Borrelia, Bordetella, Bacillus, Legionella, Pseudomonas, Mycobacterium, Helicobacter, Agrobacterium, Collectotrichum, Rhizobium, and Streptomyces) or eukaryotic (including fungal cells such as yeast cells, plant cells, protozoan cells, animal cells such as human cells and other mammalian cells such as mouse cells, rabbit cells, dog cells, etc.).

Preferably, the clonal population of cells within a pseudo-tissue sample comprise mammalian cells. Any mammalian cells (e.g., somatic cells) may be used, including blood cells (erythrocytes and leukocytes), endothelial cells, epithelial cells, neuronal cells (from the central or peripheral nervous systems), muscle cells (including myocytes and myoblasts from skeletal, smooth or cardiac muscle), connective tissue cells (including fibroblasts, adipocytes, chondrocytes, chondroblasts, osteocytes and osteoblasts) and other stromal cells (e.g., macrophages, dendritic cells, Schwann cells. Mammalian germ line cells (spermatocytes and oocytes) may also be included within a pseudo-tissue sample, as may the progenitors, precursors and stem cells that give rise to the above-described somatic and germ cells. In many cases, cells used in methods and compositions of the invention will be immortalized cells.

Cells that are suitable for inclusion within a pseudo-tissue sample may also include cells from mammalian tissues or organs such as cells derived from brain, kidney, liver, pancreas, blood, bone marrow, muscle, nervous, skin, genitourinary, circulatory, lymphoid, gastrointestinal and connective tissue sources, as well as those derived from a mammalian (including human) embryo or fetus.

In one embodiment of the invention, the cells of a clonal population of cells within a pseudo-tissue sample are hybrid cells. As used herein, the term “hybrid cell” is intended to mean a cell formed by combining two or more cells, e.g., by fusion. Generally, at least one of the cells involved in forming the hybrid cell is termed a “donor cell.” The donor cell may contribute al or a portion of its nucleic acid content to the resulting hybrid cell. By the term “fusing” or “fusion” of two or more cells is meant a method in which two or more cells are combined to form a single hybrid cell which contains all or part of at least the nucleic acid content of each individual cell. Fusion may be accomplished by any method of combining cells under fuseogenic conditions well known in the art (See, for example, Harlow & Lane in Antibodies, Cold Spring Harbor Press, New York (1988)). Known methods for fusing cells include the use of polyethylene glycol (PEG) or Sendai virus.

The hybrid cells used in methods and present in compositions of the invention may contain only one or a few chromosomes from the donor cell. Alternatively, the hybrid cells used in methods and present in compositions of the invention may contain only a fragment of a chromosome from the donor cell. The chromosome fragments may be generated by any method known to those skilled in the art, including irradiating the donor cell prior to cell fusion. Hybrid cells produced from irradiated donor cells are referred to as “radiation hybrid” cells. (Cox, D. R., et al., Science 250:245-250 (1990)).

By using hybrid cells, an investigator is able correlate the particular phenotype observed in a hybrid cell with the genetic material contributed by the donor cell. For example, where a certain subcellular feature is observed in a hybrid cell but not in the corresponding non-hybrid cell (i.e., the cell containing all of the genetic material of the hybrid cell except for that which was contributed by the donor cell), the observed phenotype in the hybrid cell in many cases will be due to the expression of the genetic material that was contributed from the donor cell.

Hybrid cells which may be used in methods and compositions of the invention include hybrid cells wherein substantially all of one, two, three, four, five, six, seven, eight, nine, ten, eleven, twelve, thirteen, fourteen, fifteen, twenty, etc. chromosomes were contributed by one or more (e.g., one, two, three, four, five, six, seven, eight nine, ten, fifteen, etc.) donor cells during formation (i.e., are of donor cell origin). Additional hybrid cells which may be used in methods and compositions of the invention include hybrid cells wherein about 2%, from about 5%, from about 7%, from about 10%, from about 12%, from about 15%, from about 20%, from about 25%, from about 30%, from about 35%, from about 40%, from about 45%, from about 50%, etc. of the nuclear nucleic acid was contributed by one or more donor cells during formation of the hybrid cells. Further hybrid cells which may be used in methods and compositions of the invention include hybrid cells wherein one or more (e.g., one, two, three, four, five, six, seven, eight nine, ten, fifteen, etc.) mitochondrion was contributed by one or more donor cells during formation of the hybrid cells. In specific embodiments, hybrid cells which may be used in methods and compositions of the invention include hybrid cells wherein from about 50 megabases to about 500 megabases, from about 100 megabases to about 500 megabases, from about 200 megabases to about 500 megabases, from about 50 megabases to about 100 megabases, from about 50 megabases to about 200 megabases, from about 50 megabases to about 400 megabases, etc. of nuclear nucleic acid (e.g., DNA) was contributed by one or more donor cells during formation of the hybrid cells.

All of the cells of a clonal population of cells within a pseudo-tissue sample may be fixed cells. Alternatively, only a subset of the cells of a clonal population of cells within a pseudo-tissue sample may be fixed. In particular embodiments, greater than 70% of the cells within a pseudo-tissue sample are fixed cells. If the cells are fixed, they can be fixed according to any method known to those skilled in the art. For example, the cells can be fixed by contacting (e.g., immersing, covering, incubating or suspending) the cells in a solution comprising a fixative agent. Examples of fixative agents that can be used are aldehyde fixatives such as formaldehyde, formalin or formol, glyoxal, glutaraldehyde, hydroxyadipaldehyde, crotonaldehyde, methacrolein, acetaldehyde, pyruic aldehyde, malonaldehyde, malialdehyde, and succinaldehyde; chloral hydrate; diethylpyrocarbonate; alcohols such as methanol and ethanol; acetone; lead fixatives such as basic lead acetates and lead citrate; mercuric salts such as mercuric chloride; formaldehyde sublimates; sublimate dichromate fluids; chromates and chromic acid; and picric acid.

An exemplary method that can be used to fix cells for inclusion within a pseudo-tissue sample is as follows: The cells of a clonal population of cells are harvested and resuspended in Ca²⁺/Mg²⁺-free phosphate buffered saline (PBS) at a ratio of about 25 ml of PBS per 1 ml of a harvested cell pellet. The resuspended cells are then incubated with gentle shaking or rocking for about 20 minutes. Next, the cells are removed from the PBS by, e.g., centrifugation, and resuspended in a solution of 10% neutral buffered formalin at a ratio of about 25 ml of formalin solution per 1 ml of harvested cell pellet. The cells are incubated in the formalin solution for about 30 minutes with gentle shaking or rocking. The cells can be stored in the formalin solution at room temperature for several weeks until needed for further processing.

The skilled artisan would recognize the foregoing as just one exemplary method that can be used to fix cells. It would be understood that other methods known in the art may be used, and that, if the above-described exemplary method were used, the specific details of the method could be easily modified or altered to suit individual experimental circumstances.

Fixation can also be accomplished with high-intensity, high-frequency, non-destructive, wide-band ultrasound. (Chu, W-S, U.S. Pat. No. 6,291,180 (2001)). Heat may be used to fix tissue specimens by boiling the specimens in physiologic sodium chloride solution or distilled water for two to three minutes. Whichever fixation method is ultimately employed, the cellular strictures of the cells should be sufficiently hardened before they are embedded in a medium such as paraffin.

The cells of a clonal population of cells within a pseudo-tissue sample are preferably fixed cells that have been collected into an aggregation. As used herein, the term “aggregation” is intended to mean any gathering, assemblage, collection or accumulation of two or more cells, wherein the distance between adjacent cells in the aggregation is less than about twice the diameter of a single cell. Preferably, the distance between adjacent cells will be less than about one-half of the diameter of a single cell. Even more preferably, the cells within an aggregation will be in contact with, or substantially in contact with adjacent cells.

The cells may be collected into an aggregation by any method known in the art for bringing individual cells into close proximity with one another. Exemplary methods for collecting cells into an aggregation include centrifugation, filtration or mechanical compression.

The pseudo-tissues of the invention comprise aggregations of fixed cells that are embedded within an embedding medium. Examples of suitable embedding media that can be used in the context of the present invention include paraffin or other waxes, gelatin, agar, polyethlene glycols, polyvinyl alcohol, celloidin, nitrocelluloses, methyl and butyl methacrylate resins or epoxy resins which are polymerized after they infiltrate the specimen. Water soluble embedding media such as polyvinyl alcohol, carbowax (polyethylene glycols), gelatin, and agar, may also be used. Water-insoluble embedding media such as paraffin and nitrocellulose generally require that cells be dehydrated in several changes of solvent such as ethyl alcohol, acetone, or isopropyl alcohol and then be immersed in a solvent in which the embedding medium is soluble.

Standard histological techniques that are generally used in the context of embedding tissue samples (e.g., non-pseudo-tissue samples) can be adapted by the skilled artisan in order to produce the pseudo-tissue samples of the invention. That is, in the context of pseudo-tissue samples, an aggregation of fixed cells can be embedded in an embedding medium using the same or similar techniques that are known in the art for embedding standard biological tissue samples. (See, e.g., Zeller, R., Curr. Protocols Mol. Biol. pp. 14.1.1-14.1.8 (1989); Osborne, M. and Isenberg, S., Cell Biol.: A Laboratory Handbood 2^{nd} Ed., Vol. 2, pp. 486-492 (1998)). Automated histological methods for embedding samples may also be employed in the preparation of pseudo-tissue samples. Automated histological embedding methods and devices are known to those skilled in the art. (See, e.g., Thiem et al., U.S. Pat. No. 6,080,365 (2000); Ahlqvist, U.S. Pat. No. 5,312,758 (1994)).

Histological Specimens

In another aspect of the invention, histological specimens obtained from pseudo-tissue samples are provided. The histological specimens according to this aspect of the invention may be comprised of an entire pseudo-tissue sample, or, alternatively, may be a sub-portion of a pseudo-tissue sample. As used herein, the term “sub-portion” is intended to mean a fragment or piece comprising less than an entire pseudo-tissue sample. Preferred sub-portions for use in conjunction with the present invention include thin sections or cores of pseudo-tissue samples. As used herein, histological specimens comprising sub-portions (e.g., thin sections or cores) of pseudo-tissue samples may be regarded, themselves, as “pseudo-tissue samples.” In any event, the histological specimens of the invention will be in a form that facilitates the analysis of subcellular features using, e.g., microscopic visualization.

The present invention also provides methods for preparing histological specimens from pseudo-tissue samples. In an exemplary embodiment, methods are provided comprising: (a) obtaining a pseudo-tissue sample; and (b) processing said pseudo-tissue sample so that said pseudo-tissue sample is suitable for microscopic analysis.

The phrase “processing a pseudo-tissue sample,” as used herein, is intended to mean any manipulation or modification of the physical attributes of a pseudo-tissue sample such that the pseudo-tissue sample, after said manipulation or modification, will permit the microscopic visualization or analysis of the cells (including the subcellular features of the cells) within a pseudo-tissue sample. Such processing may comprise removing a sub-portion from an entire pseudo-tissue sample, for example, removing a thin section or core of a pseudo-tissue sample.

Accordingly, in a preferred embodiment of this aspect of the invention, the histological specimen will comprise a sub-portion of a pseudo-tissue sample, wherein said sub-portion is a thin section obtained from a pseudo-tissue sample. As used herein, the term “thin section” is intended to mean a sub-portion of a pseudo-tissue sample that is obtained by slicing a pseudo-tissue sample. The slicing of specimens into thin sections, using a variety of cutting methods, is well known to those skilled in the art. For example, thin sections can be prepared using a sliding microtome or a razor blade.

In another preferred embodiment, the histological specimen of the invention will comprise a core, or a thin section of a core, obtained from a pseudo-tissue sample. As used herein, the term “core” is intended to mean a portion of a pseudo-tissue sample that is obtained by punching a portion out of a pseudo-tissue sample. Instruments and methods for punching cores from specimens are well known to those skilled in the art.

The histological specimen obtained from a pseudo-tissue sample, regardless of whether it is a thin section, core, thin section of a core or other sub-portion of a pseudo-tissue sample, will generally have a thickness that facilitates the analysis of subcellular features within the cells of the specimen using, e.g., microscopic visualization. In particular embodiments, the histological specimen will have a thickness of between 2-50 μm, 5-50 μm, 10-50 μm, 20-50 μm, 2-10 μm, 2-20 μm, 2-30 μm, etc. In other particular embodiments, the histological specimen will have a thickness of between 1-2 μm.

The histological specimens of the invention (including histological specimens comprising pseudo-tissue samples and histological specimens comprising non-pseudo-tissue samples) may be of any shape suitable for microscopic analysis. Preferably, the histological specimens will have a circular, oval, square or rectangular cross-section. When the cross-section of the histological specimen is circular, the diameter of said cross-section will preferably be between 0.1 mm to about 10.0 mm, between 0.2 mm to about 5.0 mm, between 0.4 mm to about 4.0 mm, or between 0.6 mm to about 2.0 mm. Similarly, when the cross-section of the histological specimen is square or rectangular, the length of a side of said cross-section will be between from about 0.1 mm to about 10.0 mm, from about 0.2 mm to about 5.0 mm, from about 0.4 mm to about 4.0 mm, or from about 0.6 mm to about 2.0 mm.

Histological Specimen-Substrate Compositions

According to another aspect of the invention, histological specimen-substrate compositions are provided. The histological specimen-substrate compositions according to this aspect of the invention may be comprised of: (a) a planar or substantially planar substrate, and (b) one or more histological specimens, each histological specimen comprising a pseudo-tissue sample or sub-portion (e.g., a thin section or a core) of a pseudo-tissue sample; wherein said one or more histological specimens are preferably in contact with only one side of said substrate.

The planar or substantially planar substrate according to this aspect of the invention can be any suitable surface upon which histological specimens or cells can be placed for visual inspection and/or analysis, preferably for microscopic analysis. In general, the substrate will be transparent or translucent. Preferably, the substrate will be a glass slide; however, it will be understood by those skilled in the art that a variety of other materials, such as plastic or synthetic polymers can also be used in conjunction with the present invention. The substrate may be solid or semi-solid. Examples of semi-solid substrates that can be used with the invention include substrates comprised of agar, polyacrylamide, gelatin or wax. All of the histological specimens of the histological specimen-substrate composition will preferably be in contact with only one side of the substrate.

Arrays

When multiple histological specimens are included within the histological specimen-substrate compositions of the invention, as described supra, each histological specimen is preferably applied to a different, discrete location on the substrate relative to the other histological specimens. Further, in many embodiments, the specimens will not overlap with one another on the substrate.

The histological specimen-substrate compositions according to the invention may comprise a plurality of histological specimens, wherein each histological specimen comprises a pseudo-tissue sample and/or non-pseudo-tissue sample. For example, the histological specimen-substrate composition may contain from about 1 to about 1000 histological specimens. The histological specimen-substrate compositions according to this aspect of the invention may, alternatively, comprise from about 2 to about 10, from about 2 to about 20, from about 2 to about 30, from about 2 to about 40, from about 2 to about 50, from about 2 to about 60, from about 2 to about 70, from about 2 to about 80, from about 2 to about 90, from about 2 to about 100, from about 2 to about 120, from about 2 to about 150, from about 2 to about 200, from about 2 to about 250, from about 2 to about 260, from about 2 to about 270, from about 2 to about 280, from about 2 to about 290, from about 2 to about 300, from about 2 to about 350, from about 2 to about 400, from about 2 to about 500, from about 2 to about 1000, from about 5 to about 10, from about 5 to about 20, from about 5 to about 30, from about 5 to about 40, from about 5 to about 50, from about 5 to about 60, from about 5 to about 70, from about 5 to about 80, from about 5 to about 90, from about 5 to about 100, from about 5 to about 120, from about 5 to about 150, from about 5 to about 200, from about 5 to about 250, from about 5 to about 260, from about 5 to about 270, from about 5 to about 280, from about 5 to about 290, from about 5 to about 300, from about 5 to about 350, from about 5 to about 400, from about 5 to about 500, from about 10 to about 20, from about 10 to about 30, from about 10 to about 40, from about 10 to about 50, from about 10 to about 60, from about 10 to about 70, from about 10 to about 80, from about 10 to about 90, from about 10 to about 100, from about 10 to about 120, from about 10 to about 150, from about 10 to about 200, from about 10 to about 250, from about 10 to about 260, from about 10 to about 270, from about 10 to about 280, from about 10 to about 290, from about 10 to about 300, from about 10 to about 350, from about 10 to about 400, from about 10 to about 450, from about 10 to about 500, from about 20 to about 30, from about 20 to about 40, from about 20 to about 50, from about 20 to about 60, from about 20 to about 70, from about 20 to about 80, from about 20 to about 90, from about 20 to about 100, from about 20 to about 120, from about 20 to about 150, from about 20 to about 200, from about 20 to about 250, from about 20 to about 260, from about 20 to about 270, from about 20 to about 280, from about 20 to about 290, from about 20 to about 300, from about 20 to about 350, from about 20 to about 400, from about 20 to about 500, from about 30 to about 40, from about 30 to about 50, from about 30 to about 60, from about 30 to about 70, from about 30 to about 80, from about 30 to about 90, from about 30 to about 100, from about 30 to about 120, from about 30 to about 150, from about 30 to about 200, from about 30 to about 250, from about 30 to about 260, from about 30 to about 270, from about 30 to about 280, from about 30 to about 290, from about 30 to about 300, from about 30 to about 350, from about 30 to about 400, from about 30 to about 500, from about 40 to about 50, from about 40 to about 60, from about 40 to about 70, from about 40 to about 80, from about 40 to about 90, from about 40 to about 100, from about 40 to about 120, from about 40 to about 150, from about 40 to about 200, from about 40 to about 250, from about 40 to about 260, from about 40 to about 270, from about 40 to about 280, from about 40 to about 290, from about 40 to about 300, from about 40 to about 350, from about 40 to about 400, from about 40 to about 500, from about 50 to about 450, from about 100 to about 450, or from about 200 to about 450 histological specimens.

The histological specimen-substrate composition according to this aspect of the invention, when it comprises multiple (e.g., from about 2 to about 1000) histological specimens comprising pseudo-tissue samples, will often be constructed such that each, or approximately each of the histological specimens comprise a distinct pseudo-tissue sample not represented by any other histological specimen located on said histological specimen-substrate composition. In particular embodiments, the histological specimen-substrate composition will comprise multiple, distinct pseudo-tissue samples (e.g., histological specimens prepared from pseudo-tissue samples), each applied to a different discrete location on the substrate in an ordered two-dimensional array.

Histological specimen-substrate compositions of the invention may include control histological samples. For instance, a control histological sample may be a sample that exhibits a known phenotypic trait or possesses a particular known subcellular feature. The known phenotypic trait or subcellular feature of the control sample can be compared with the phenotypes and subcellular features possessed by other histological specimens of the histological specimen-substrate composition. The control histological sample can be a histological sample prepared by any method known to those skilled in the art; however, in certain embodiments, the control sample will be a pseudo-tissue sample comprising cells with a known phenotypic trait or subcellular feature. To or more loci on the histological specimen-substrate composition may contain the same control histological sample.

The invention also provides methods for preparing an array of histological specimens, wherein each histological specimen comprises a pseudo-tissue sample. The methods according to this aspect of the invention comprise: (a) obtaining two or more (e.g., from about 2 to about 1000) pseudo-tissue samples; (b) processing each pseudo-tissue sample so that each pseudo-tissue sample is suitable for microscopic analysis; and (c) placing said processed pseudo-tissue samples on a planar or substantially planar substrate; thereby producing an array of histological specimens. The processing of the pseudo-tissue samples according to this aspect of the invention may comprise, e.g., removing a sub-portion such as a thin section or core from each pseudo-tissuc sample.

In a preferred embodiment of this aspect of the invention, from about 50 to about 450 pseudo-tissue samples are obtained, each containing a distinct clonal population of cells. A thin section or core is then removed from each pseudo-tissue sample, thereby producing a histological specimen corresponding to each pseudo-tissue sample. Next, the thin sections or cores are applied to different, discrete locations on a substrate such as a standard glass microscope slide, thereby producing an array of histological specimens.

It will be understood by those skilled in the art that the above preferred method can be varied substantially to produce an array of histological specimens. Alternative methods can also be used. For example, in one alternative method, multiple (e.g., from about 2 to about 1000) histological specimens, each obtained from a distinct pseudo-tissue sample, are brought into a “recipient” block of embedding medium. The resulting multi-specimen array block is then sliced into one or more sections that can be transferred to glass slides or other substrates. Methods that have been described in the art for producing arrays of tissue samples (See, e.g., Schraml, P., et al., Clinical Cancer Res. 5:1966-1975 (1999); Koonen, J., et al., Nat. Med. 4:844-847 (1998); U.S. Pat. No. 6,103,518 (2000)) can be modified by the skilled artisan for purposes of producing an array of pseudo-tissue samples.

It will also be understood by those skilled in the art that the histological specimen-substrate compositions and arrays of the invention may comprise, in addition to histological specimens that comprise pseudo-tissue samples, traditional histological specimens comprised of non-pseudo-tissue samples (e.g., biopsy samples from animal tissue)

Methods for Analyzing Tissues

In another aspect of the invention, methods for analyzing a tissue sample are provided. Methods according to this aspect of the invention include those which involve, for example, comparing a candidate histological specimen to a reference histological specimen, wherein the candidate histological specimen to be analyzed comprises either (1) a tissue sample or (2) a pseudo-tissue sample which comprises a clonal population of cells, and the reference histological specimen comprises a pseudo-tissue sample which comprises a clonal population of cells. According to this aspect of the invention, one or more subcellular features that are common to both the candidate and the reference histological specimens are detected. By identifying subcellular features in the candidate specimen that are also present in the reference specimen, the researcher is able to correlate the phenotype of the candidate specimen with the genotype of the reference specimen, i.e., the genotype of the clonal population of cells within the pseudo-tissue sample.

According to one embodiment of this aspect of the invention, a method is provided comprising: (a) obtaining one or more reference histological specimens, each reference histological specimen comprising a pseudo-tissue sample; (b) obtaining one or more candidate histological specimens, each candidate histological specimen to be analyzed comprising either (1) a tissue sample or (2) a pseudo-tissue sample which comprises a clonal population of cells; and (c) detecting on or more subcellular features common to said reference and candidate histological specimens.

According to another embodiment of this aspect of the invention, a method is provided for analyzing a plurality of tissue samples, said method comprising: (a) obtaining one or more reference histological specimens, each reference histological specimen comprising a pseudo-tissue sample; (b) obtaining a plurality of candidate histological specimens, each candidate histological specimen to be analyzed comprising either (1) a distinct tissue sample or (2) a pseudo-tissue sample which comprises a clonal population of cells; and (c) detecting one or more subcellular features common to said reference and candidate histological samples.

According to yet another embodiment of this aspect of the invention, a method is provided for identifying a tissue sample that possesses a pre-selected subcellular feature, said method comprising: (a) obtaining one or more reference histological specimens, each reference histological specimen comprising a pseudo-tissue sample or sub-portion thereof; (b) obtaining a plurality of candidate histological specimens, each candidate histological specimen to be analyzed for the presence of said pre-selected subcellular feature comprising either (1) a distinct tissue sample or (2) a pseudo-tissue sample which comprises a clonal population of cells; and (c) detecting said pre-selected subcellular feature in said candidate reference histological specimens.

As used herein, the term “candidate histological specimen” is intended to mean a histological specimen to be analyzed comprising either (1) a tissue sample or (2) a pseudo-tissue sample which comprises a clonal population of cells. The term “reference histological specimen” is intended to mean a histological specimen comprising a pseudo-tissue sample or sub-portion thereof.

According to this aspect of the invention, the pseudo-tissue sample of the reference histological specimen may be a pseudo-tissue sample that has been subjected to further processing so that said pseudo-tissue sample is suitable for microscopic analysis. As used herein, the term “further processing” is intended to mean any modification, manipulation or treatment known by those skilled in the histological arts that can be applied to a pseudo-tissue sample in order to facilitate the microscopic visualization and/or analysis of subcellular features within the sample. Examples of such further processing include, e.g., removing a sub-portion from said pseudo-tissue sample. The sub-portion that is removed may be, e.g., a thin section or core of a pseudo-tissue sample.

The candidate histological specimens of the invention can be any histological specimen obtained by any histological preparation method known to those skilled in the art or described herein. In one embodiment, the histological specimens are sections of frozen tissue or cells. The histological specimens can also be a smear; e.g., a liquid specimen, or small solid chunks of tissue suspended in liquid, that is smeared on a microscope slide.

Although the candidate histological specimens of this aspect of the invention will typically be traditional histological specimens comprised of tissue samples (as described in detail infra), it is envisioned that, in some embodiments of the invention, the candidate histological specimen, like the reference histological specimens, will comprise pseudo-tissue samples or sub-portions thereof.

Preferably, the candidate histological specimens are thin sections or cores prepared from paraffin-embedded fixed tissue. Where the embedding medium is paraffin, suitable solvents for the paraffin are xylene, toluene, benzene, petroleum, ether, chloroform, carbon tetrachloride, carbon bisulfide, and cedar oil. Preferably a tissue specimen is immersed in two or three baths of the paraffin solvent after the tissue is dehydrated and before the tissue specimen is embedded in paraffin.

The candidate histological specimens of the present invention can be prepared according to the following exemplary method: First, the tissue sample is immersed in a fixative such as formalin solution. The fixation step functions to stop the process of decay and to stabilize the tissue so as to protect it against the physical and chemical rigors of subsequent processing steps. Second, the fixed specimen is immersed in a dehydrating agent. The dehydrating agent serves to remove some or all of the free water contained in the specimen. Exemplary dehydrating agents include ethanol, methanol, isopropanol, and mixtures thereof. (Hoffmann, R. W., et al., U.S. Pat. No. 6,017,725 (2000)). Third, the specimen is immersed in a clearing agent. The clearing agent is a solvent, such as xylene or toluene, that displaces the dehydrating agent. Finally, the cleared specimen is immersed in a bath of paraffin which impregnates the specimen and permits it to be sliced into thin sections for subsequent mounting onto slides. The paraffin may optionally be dissolved from the tissue followed by restoration of water to the sections. (See generally, Lillie, R. D., et al. “Histopathologic Technic and Practical Histochemistry” (4th Ed.), McGraw-Hill (1976), for details of fixing, slicing, embedding, and staining specimens).

The tissue sample to be analyzed according to the present invention (i.e., the tissue sample within the candidate histological specimen) can be tissue obtained from any biological source. For example, the tissue sample may be from an animal such as a rodent. Preferably, the tissue sample is from a human. The tissue sample may be from normal or diseased tissue. In one embodiment, the tissue is obtained from a tumor.

The present invention is also directed to the detection of one or more subcellular features within candidate and reference histological specimens, e.g., within the cells of a pseudo-tissue sample. As used herein, the term “subcellular feature” is intended to mean any observable characteristic of a cell.

According to the present invention, subcellular features within a histological specimen can be detected using any method of detection known by those skilled in the art. For example, subcellular features may be detected by contacting the candidate and reference histological specimens with a reagent that facilitates the detection of one or more subcellular features. The specific reagent used will depend upon the subcellular feature sought to be detected. The appropriate reagent in any given situation will be known to the skilled artisan.

Exemplary reagents that facilitate the detection of one or more subcellular features include antibodies, nucleic acid probes, compounds capable of detecting carbohydrates, and compounds capable of detecting enzymatic activity. Such reagents may be detectably labeled. In addition, the reagents that facilitate the detection of one or more subcellular features may include dyes or other compounds that impart an artificial color to various regions and components within cells. Commonly used dyes that can be used to detect subcellular features in histological specimens according to the present invention include hematoxylin, Haematoxylon campechianum, and cosin.

Exemplary subcellular features that can be detected according to the present invention include morphological characteristics such as cellular morphology, nuclear morphology, morphology of the cndoplasmic reticulum, mitochondrial morphology, and cytoskeletal morphology. Cytoskeletal morphology may be identified by analyzing tubulin, actin, or any other cytoskeletal component. The observation and/or detection of morphological characteristics can be accomplished by methods well known to those in the art.

Additional subcellular features that can be detected according to the present invention include proteins and other polypeptides. Proteins and polypeptides can be detected, for example, by using an antibody that binds specifically to a protein or polypeptide in the histological specimen. The antibody used according to this aspect of the invention may be directly or indirectly labeled. The label may be any detectable label including a fluorescent label or radiolabel. A variety of labeling substances are described in the patent and non-patent literature. These include fluorophores, radioisotopes, enzymes, dyes, enzyme cofactors, enzyme inhibitors, luminescent materials, ferritin, colloidal gold, etc. (Langone, et al., eds., Methods in Enzymology, volume 70, N.Y.: Academic Press (1980)). Typical fluorophores include fluorescein, rhodamine, phycoerythrin, or phycocyanin. Methods of detecting proteins using antibodies are well known to those skilled in the art.

Also included among the subcellular features that can be detected according to the present invention are nucleic acids. Nucleic acids can be detected, for example, by using a nucleic acid probe that binds to a specific nucleotide sequence. The nucleic acid probes used according to this aspect of the invention may be directly or indirectly labeled. The label may be any detectable label, including a fluorescent label or radiolabel. A variety of labeling substances are described in the patent and non-patent literature. These include fluorophores, radioisotopes, enzymes, dyes, enzyme cofactors, enzyme inhibitors, luminescent materials, ferritin, colloidal gold, etc. (Langone, et al., eds., Methods in Enzymology, volume 70, N.Y.: Academic Press (1980)). Typical fluorophores include fluorescein, rhodamine, phycoerythrin, or phycocyanin. Methods of detecting nucleic acids with nucleic acid probes are well known to those skilled in the art.

Another exemplary subcellular feature that can be detected according to the present invention is carbohydrate. Methods for detecting a carbohydrate within a cell are well known to those skilled in the art.

Another exemplary subcellular feature that can be detected according to the present invention is enzymatic activity. Any enzyme whose reaction products or intermediates can be detected or identified is included within this aspect of the invention. Enzymatic activity may be measured by several methods including zymography and the use of specific substrates, including fluorometric, colorometric, and radioactive substrates. (Bonner et al., U.S. Pat. No. 6,251,516 (2001)). Methods for detecting the activities of many cellular enzymes are well known to those skilled in the art.

The method of the invention involves, inter alia, detecting subcellular features that are common to the candidate and reference histological specimens. In order to facilitate the identification of common subcellular features, a candidate histological specimen and a reference histological specimen can be placed on a common substrate. For example, a candidate histological specimen and a reference histological specimen can be placed in close vicinity to one another on the same microscope slide, thereby permitting a side-by-side comparison of the subcellular features of the specimens. Aside from a microscope slide, any substrate or support can be used that does not interfere with the detection of subcellular features. Examples of suitable substrates are described elsewhere herein.

In one embodiment of the invention, a plurality of candidate histological specimens and one or more reference histological specimens are arranged in an ordered array. The ordered array will preferably facilitate automated analysis of the specimens. Methods for preparing ordered arrays of tissue samples, also known as “tissue arrays” or “tissue microarrays,” have been described. (Leighton, S. B., U.S. Pat. No. 6,103,518 (2000)); (Battifora, H. A., U.S. Pat. No. 4,820,504 (1989)); (Bubendorf, L., et al., Cancer Res. 59:803-806 (1999)); (Schraml, P, et al., Clinical Cancer Res. 5:1966-1975 (1999)).

Subcellular features that are common to the candidate and reference histological specimens may be detected according to a number of methods known to those skilled in the art. For example, visual methods may be used. In a preferred embodiment, a candidate histological specimen and a reference histological specimen are placed on a single microscope slide. According to this embodiment, the subcellular features of both the candidate and reference specimens are visually inspected. Particular subcellular features that are found in both the candidate and the reference specimen can then be identified and their commonality noted.

In addition to visual methods, automated detection methods can be used in conjunction with the present invention. Automated histological classification methods have been described. (Boon, M. E., et al., U.S. Pat. No. 5,939,278 (1999)); (Luck, R. L., et al., U.S. Pat. No. 5,257,182 (1993)); (Rutenberg, M. R., U.S. Pat. No. 4,965,725 (1990)).

The present invention is also directed to identifying a tissue sample that possesses a pre-selected subcellular feature. According to this aspect of the invention, a reference histological specimen is used comprising a pseudo-tissue sample (including a pseudo-tissue sample that has been subjected to further processing so that said pseudo-tissue sample is suitable for microscopic analysis), said pseudo-tissue sample comprising, inter alia, a clonal population of cells, wherein the clonal population of cells is comprised of cells that possess said pre-selected subcellular feature. The pre-selected subcellular feature can be any subcellular feature that an investigator might choose to select for identification in a candidate histological specimen. The pre-selected subcellular feature may include cellular morphology, a nucleic acid, a polypeptide, a carbohydrate, or enzymatic activity.

In a preferred embodiment of this aspect of the invention, a plurality of candidate histological specimens is obtained, wherein each candidate specimen comprises a distinct tissue sample to be analyzed for the presence of a pre-selected subcellular feature. For example, two or more tissue samples, each obtained from different organisms, or from different tissue regions of a single organism, would be considered distinct tissue samples.

According to this aspect of the invention, the candidate and reference histological samples are analyzed for the presence of the pre-selected subcellular feature. A tissue sample that possesses the pre-selected subcellular feature is identified by virtue of the fact that the pre-selected subcellular feature is detected in both the reference histological sample and in the candidate histological sample comprising the particular tissue sample.

Kits

The invention also provides kits for preparing the compositions of the invention and for practicing the methods of the invention. In particular, the invention provides kits for preparing pseudo-tissue samples and histological specimen-substrate compositions which comprise pseudo-tissue samples. The invention also provides kits for identifying and/or analyzing histological specimens such as, e.g., tissue samples that have been prepared according to standard methods of histological processing in order to facilitate the identification and/or analysis of subcellular features. The invention further provides kits for preparing tissue arrays, including tissue microarrays and kits comprising tissue arrays. Examples of tissue arrays which can be provided in or prepared using kits of the invention are described elsewhere herein, but include tissue arrays containing both pseudo-tissue samples and other histological specimens.

In one embodiment, a kit is provided for preparing a pseudo-tissue sample or an array of pseudo-tissue samples. In another embodiment, a kit is provided which comprises one or more tissue arrays. Kits according to these embodiments of the invention may comprise one or more of the following: (a) one or more (e.g., one, two, three, four, five, ten, fifteen, twenty, forty, etc.) clonal population of cells, (b) a solution comprising a fixative agent, (c) one or more embedding medium, and (d) one or more tissue array; wherein said kit components can be combined to form or construct a pseudo-tissue sample, or an array of pseudo-tissue samples.

The invention also provides kits for analyzing a tissue sample. Such kits may comprise an array of reference histological specimens, each reference histological specimen comprising a pseudo-tissue sample, one or more solution comprising a fixative agent, and embedding medium.

Kits according to these embodiments of the invention may further comprise one or more additional components selected from the group consisting of: (1) one or more instrument for obtaining a thin section or core from a pseudo-tissue sample (e.g., a microtome, razor, etc.); (2) one or more substrate upon which histological specimens may be placed for microscopic analysis (e.g., glass or plastic slides or tubes, components with at least one planar or substantially planar metal, glass, or plastic surface, as well as other components with at least one other planar or substantially planar solid or semi-solid surface, etc.); (3) one or more reagent (e.g., stain, dye, antibody, nucleic acid probe, enzymatic substrate or catalyst, etc.) that facilitates the detection of one or more subcellular features (e.g., morphological features, polypeptides, nucleic acids, carbohydrate, enzymatic activity, etc.); (4) water (e.g., deionized, distilled, sterilized, etc.); (5) one or more buffers (e.g., buffers in dry, powder form or reconstituted in liquid such as water, etc.); (6) one or more containers (e.g., box, carton, vial, bottle, ampule, tube, etc.) for storing or transporting kit components; (7) computer software that facilitates the analysis (e.g., automated analysis) of an array of histological specimens; and (8) user instructions for preparing a pseudo-tissue sample or an array of pseudo-tissue samples.

As noted above, kits of the invention may contain any number of various components suitable for practicing methods of the invention. One example of such a component is instructions for performing methods of the invention. Examples of such instructions include those which direct individuals using the kits to perform methods which result in the identification of subcellular features of tissue samples. Methods related to such processes, as well as other processes, are referred to elsewhere herein.

As one skilled in the art would recognize, the full text of these instructions need not be included with the kit. One example of a situation in which kits of the invention would not contain such full length instructions is where directions are provided which inform individuals using the kits where to obtain instructions for practicing methods of the invention. Thus, instructions for performing methods of the invention may be obtain from internet web pages, separately sold or distributed manuals or other product literature, etc. The invention thus includes kits which direct kit users to locations where they can find instructions which are not directly packaged and/or distributed with the kits. These instructions may be in any form including, but not limited to, electronic or printed forms.

The following examples are illustrative, but not limiting, of the method and compositions of the present invention. Other suitable modifications and adaptations of the variety of conditions and parameters normally encountered in molecular biology and chemistry which are obvious to those skilled in the art in view of the present disclosure are within the spirit and scope of the invention.

### EXAMPLES

### Example 1

### Analysis of a Tissue Sample Expressing a Gene of Interest

The histological analysis of tissue samples is complicated by the fact that most tissue samples comprise various cell types as well as other tissue material. The manner in which tissue samples are prepared can contribute to the difficulty of their analysis. The heterogenous composition of tissue samples greatly complicates the detection of subcellular features that are of diagnostic value, such as oncogene expression in cancerous tissue. For example, high levels of expression of an oncogene may be difficult to discern in a tissue sample that comprises both normal and cancerous cells. Although the normal (non-cancerous) cells may express the oncogene to some extent, the cancerous cells will typically express the oncogene at a higher level. The differences in expression levels between the cancerous and non-cancerous cells within a given tissue sample, however, may be difficult to detect. That is, the normal cells will produce a background signal that can obscure the enhanced signal produced by the cancerous cells.

The present invention helps to alleviate problems of this sort by providing a reference histological specimen comprising a pseudo-tissue sample. The pseudo-tissue sample will often comprise, inter alia, a clonal population of cells that are well characterized and that are known to exhibit a given subcellular feature. The reference histological specimen is typically analyzed side-by-side with a candidate histological specimen which generally comprises the tissue sample of interest. Such a side-by-side analysis allows the investigator to directly compare the subcellular features within the candidate histological specimen to those found in the reference histological specimen. Thus, cancerous cells within a tissue sample of interest can be identified according to the present invention by using a reference histological specimen, (e.g., a pseudo-tissue sample comprising a clonal population of cancerous cells that is analyzed alongside the tissue sample of interest). If the cells within the tissue sample are found to exhibit a subcellular feature(s) that is identical (or nearly identical) to those features found in the clonal cells within the reference specimen, a correlation can be drawn between the phenotype of the candidate sample and the genotype of the clonal cells.

In this Example, a tissue sample is analyzed for the expression of a pre-selected gene of interest. The gene of interest can be any gene, for example, the well-studied oncogene, ERBB2. (Schraml, P., et al., Clinical Cancer Res. 5:1966-1975 (1999)). High levels of ERBB2 expression in the tissue sample may indicate neoplasticity.

First, a tissue sample is obtained from a tissue region suspected of expressing the gene of interest at high levels. For example, a biopsy can be obtained from a tissue mass that is suspected of being cancerous. Any method of biopsy known to those skilled in the art can be used to obtain the tissue sample.

A clonal population of cells is also obtained, the members of which are known to express high levels of the gene of interest. For instance, a tissue culture cell line can be used that is known to possess a mutation that causes high expression of the gene of interest. Alternatively, a cell line can be transfected with a genetic vector that causes high expression of the gene of interest. In addition to, or instead of a tissue culture cell line, a population of hybrid cells may be used. The hybrid cells may be derived from a fusion of rodent cells with irradiated human cells such that the only human-derived genetic element in the hybrid cells is a genetic element that causes high expression of the gene of interest.

Second, a pseudo-tissue sample is prepared using the clonal population of cells. The pseudo-tissue sample can be prepared, in general, by fixing the cells of the clonal population, collecting or forming the fixed cells into an aggregation, and embedding the aggregation of fixed cells in an embedding medium. The pseudo-tissue sample is then subjected to further processing (e.g., sectioning or coring) to produce a reference histological specimen.

Third, a candidate histological specimen is prepared from the tissue sample to be analyzed. The preparation of the candidate histological specimen (comprising the tissue sample) and the reference histological specimen(s) (comprising the pseudo-tissue sample) will typically be performed in parallel with one another. That is, both specimens are treated identically in the histological preparation process.

The candidate histological specimen can be prepared by any standard method. According to one method of preparation, the tissue sample is immersed in a fixative for several hours. The fixative can be any fixative such as formalin. After fixation, the samples are treated such that all or most of the water is removed and replaced with paraffin wax. The paraffin treated sample may then be embedded in a larger block of molten paraffin. After the block solidifies, a microtome can be used to slice thin sections (approximately 5 μm in thickness) from the block, thereby producing the candidate histological specimen.

Fourth, the reference and candidate specimens are placed side-by-side on a substrate such as a microscope slide.

Fifth, after the candidate and reference histological specimens are prepared, they are processed such that expression of the gene of interest can be detected. Expression of the gene of interest can be detected by using, for example, an antibody that specifically interacts with the polypeptide product of the gene of interest. Alternatively, a nucleic acid probe can be used that interacts specifically with mRNA corresponding to the gene of interest. In either case, the antibody or the nucleic acid probe will preferably be detectably labeled, for example, with a fluorescent label. The detectably labeled antibody or nucleic acid probe is allowed to contact the candidate and reference histological specimens.

If antibody detection is used, two different antibodies can be used in succession: the first, “primary” antibody will be specific to the polypeptide product of the gene of interest and will not be labeled. The second, “secondary” antibody will be specific to the primary antibody and will be labeled. The use of a primary and secondary antibody sequence will facilitate amplification of the detection signal.

The skilled artisan will appreciate that various blocking and washing steps will generally be employed to facilitate optimum specific interaction between the antibody or nucleic acid probe and their respective targets in the candidate and reference histological specimens. Methods for detecting polypeptides with antibodies, and for detecting mRNA with nucleic acid probes, are well known to those skilled in the art. Preferably, the candidate and reference specimens will be arranged on the same microscope slide and will be processed identically.

After processing the candidate and reference histological specimens with a labeled antibody or nucleic acid probe, the specimens are then analyzed. For instance, if fluorescent labels are used, the specimens can be observed using a fluorescence microscope using appropriate filters. Fluorescence microscopy techniques are well known to those skilled in the art. The images from the candidate histological specimen are compared to those from the reference histological specimen. The comparison of images may be done visually or through the use of computer-aided analysis software. The reference specimen should reveal a pattern of fluorescence that is indicative of high expression of the gene of interest. A similar or identical pattern in the candidate histological specimen will indicate that the tissue sample being analyzed is comprised of cells also exhibiting high levels of expression of the gene of interest.

As this Example illustrates, the preparation and use of a reference histological specimen according to the present invention will simplify and streamline the histological analysis of tissue samples. More specifically, the side-by-side comparison of a candidate histological specimen with a reference histological specimen can greatly aid in the identification of subcellular features within a tissue sample that are of substantial diagnostic value, such as the identification of high levels of expression of an oncogene indicative of cancer.

### Example 2

### Analysis and Comparison of Tissue Samples and Pseudo-Tissue Samples

Introduction

Recently, tissue arrays (including tissue microarrays) have been described and characterized for their ability to successfully screen substantial numbers of tissues at one time. With tissue arrays, molecular and cell biology can be combined with the analysis of DNA, RNA, and protein expression through immunohistochemistry and in situ hybridization techniques. The visualization of morphological characteristics and the identification of molecular markers within a tissue has proven extremely effective in the in vivo study of a variety of diseases including cancer.

As more and more has been learned about tissues and their specificity in disease, it has become apparent that the individual cells that make up tissues are extremely important. Cellular protein and gene expression can play a key role in how a particular protein or plurality of proteins is utilized within a cell and how loss of gene regulation can negatively effect a cell and the surrounding tissue. Gene and protein expression can even be cell specific. Antibody labeling and in situ hybridization can show patterns of protein expression within the cell. Localized expression can be observed, e.g., in the nucleus and in the cytosol. The expression of membrane-bound or secreted proteins can also be observed and/or monitored using the same antibody labeling and in situ labeling techniques. Such information regarding the expression of proteins can be very helpful in understanding the dynamics of a protein with a cell. Once the pattern of protein expression is understood, observing that expression in various tissues can help the investigator better understand the dynamics of cells and tissue interactions.

Pseudo-tissue samples can be used to further understand individual cellular interactions. This Example provides an illustration of one practical application of pseudo-tissue technology. Additionally, this Example demonstrates how specific immortalized cells from specific tissues may be included within pseudo-tissue samples to confirm cellular specificity within a tissue.

Materials and Methods

Histological specimen-substrate compositions are prepared as described elsewhere herein. The histological specimen-substrate compositions may comprise multiple tissues that are arrayed alongside multiple pseudo-tissue samples. Each pseudo-tissue sample may comprise a different cell type. An antibody specific to breast tissue (anti-BRST-2) can be used to confirm staining of breast cells through immunohistochemistry (IHC). Negative control tissues and pseudo-tissue samples can also be included to confirm the specificity of the reaction. Examples of the types of tissues and pseudo-tissue samples that can be analyzed according to the present invention are shown in Table I.

TABLE I

| Tissue Sample | Pseudo-Tissue Sample |
|---|---|
| Breast (+) | Breast - T47D (+) |
| Ovary (−) | Breast - MCF7 (+, −) |
| Colon (−) | Breast - Hs578T (+, −) |
| Liver (−) | Colon - Colo205 (−) |
| Prostate (−) | Ovary - OVCAR (−) |

Postive (+) denotes the positive controls, while Negative (−) denotes the negative controls.

For this Example, therefore, histological specimen-substrate compositions (also referred to in this Example as “the samples”) comprising both conventional tissue samples and pseudo-tissue samples are used. The histological specimens are arranged in an array on microscope slides. The reagents that may be used in this Example include: Antibody GCDFP-15 (Signet BRST-2 pre-diluted 611-23); Antibody Diluent (DAKO S0809); Citrate Buffer pH 6.0 (Invitrogen Corp.); DI H₂O; Wash buffer (TBS/0.03% TWEEN™ 20 (pH 7.6) DAKO #S3001); Graded EtOH; Xylene and Immunohistochemistry kit components (Peroxide block, Blocking agent, Primary antibody, Universal secondary antibody (linker), Strepavidin HRP reagent, DAB chromogen substrate, and DAB substrate).

The prepared histological specimen-substrate compositions are next dried for 1 hour at 55° C. and then washed, first in xylene 2×10 min under a hood (200 ml/container), then in graded alcohols (100% for 2 min, 100% for 2 min, 95% for 2 min, 95% for 2 min, 80% for 2 min, in a 200 ml/container), and finally in DI H₂O (200 mls/container) for 2 min.

After the washing steps, the histological specimen-substrate compositions are dipped in steamed DI water. The samples are then incubated in peroxides solution for 10 min and then washed in Wash buffer ˜5 min. The tissue samples and pseudo-tissue samples within the histological specimen-substrate compositions are blocked with kit blocking agent or 10% FCS in PBS or TBS ˜5 min. The samples are washed again in Wash buffer before applying enough primary antibody to cover the samples (˜100-250 ul).

After application of theprimary antibody, the samples are incubated at RT ˜30 min and then rinsed in Wash buffer for 5 min. The secondary (linker) antibody is then applied for 10 min, and the samples are rinsed in Wash buffer for 5 min. Next, HRP reagent is applied to the samples and the samples are incubated for 0 min. Following the 10 minute incubation, the samples are rinsed in Wash buffer for 5 min.

DAB chromogen and DAB substrate are combined according to the kit directions and the reagent is applied to the samples and allowed to incubate 1-15 min. The tissue colors are visually observed to determine when to stop the reactions. After the appropriate color is observed, the samples are rinsed in Wash buffer for 5 min. The samples are then dipped in hematoxylin for 10 seconds and washed in running tap water for 2-5 min.

Lastly, the samples are dehydrated through graded alcohols (80% gentle agitation/2 changes, 95% gentle agitation/2 changes, 100% gentle agitation/2 changes) and are then subjected to a Xylene wash (gentle agitation/2 changes). A coverslip is then applied using mounting medium. The tissues and pseudo-tissue samples can be viewed under a microscope in order to identify and compare, side-by-side, the subcellular features within the pseudo-tissue samples and the tissue samples.

Although the foregoing invention has been described in some detail by way of illustration and example for purposes of clarity of understanding, this invention is not limited to the particular embodiments disclosed, but is intended to cover all changes and modifications that are within the spirit and scope of the invention as defined by the appended claims.

All publications and patents mentioned in this specification are indicative of the level of skill of those skilled in the art to which this invention pertains. All publications and patents are herein incorporated by reference to the same extent as if each individual publication or patent application were specifically and individually indicated to be incorporated by reference.

What is claimed is:

1. A pseudo-tissue sample comprising an embedding medium and a clonal population of cells, wherein said clonal population of cells comprise fixed cells that have been collected into an aggregation and are embedded within said embedding medium.

2. A pseudo-tissue sample comprising an embedding medium and two or more clonal populations of cells, wherein said two or more clonal populations of cells comprise fixed cells that have been collected into a single aggregation and are embedded within said embedding medium.

3. The pseudo-tissue sample of claim 1, wherein said fixed cells are fixed mammalian cells.

4. The pseudo-tissue sample of claim 1, wherein said fixed cells are fixed hybrid cells.

5. The pseudo-tissue sample of claim 4, wherein said fixed hybrid cells are fixed radiation hybrid cells.

6. The pseudo-tissue sample of claim 1, wherein said fixed cells are formaldehyde-, formalin-, glutaraldehyde-, methanol-, or ethanol-fixed cells.

7. The pseudo-tissue sample of claim 6, wherein said fixed cells are formalin-fixed cells.

8. The pseudo-tissue sample of claim 1, wherein said embedding medium comprises an ingredient selected from the group consisting of paraffin, gelatin, agar, wax, polyethylene glycol, polyvinyl alcohol, celloidin, nitrocellulose, methyl and butyl methacrylate resins, and epoxy resins.

9. The pseudo-tissue sample of claim 8, wherein said embedding medium comprises paraffin.

10. A histological specimen comprising a pseudo-tissue sample, said pseudo-tissue sample comprising an embedding medium and a clonal population of cells, wherein said clonal population of cells comprise fixed cells that have been collected into an aggregation and are embedded within said embedding medium.

11. A histological specimen comprising a sub-portion of a pseudo-tissue sample, said pseudo-tissue sample comprising an embedding medium and a clonal population of cells, wherein said clonal population of cells comprise fixed cells that have been collected into an aggregation and are embedded within said embedding medium.

12. The histological specimen of claim 11, wherein said sub-portion is a thin section of a pseudo-tissue sample.

13. The histological specimen of claim 11, wherein said sub-portion is a core of a pseudo-tissue sample.

14. The histological specimen of claim 10, wherein said pseudo-tissue sample has a thickness of between 0.5 μm and 5.0 μm.

15. The histological specimen of claim 10, wherein said pseudo-tissue sample has a thickness of between 1.0 μm and 2.0 μm.

16. The histological specimen of claim 10, wherein said pseudo-tissue sample has a circular cross-section.

17. The histological specimen of claim 16, wherein the diameter of said circular cross-section is from about 0.6 mm to about 2.0 mm.

18. The histological specimen of claim 10, wherein said pseudo-tissue sample has a square or rectangle cross-section.

19. The histological specimen of claim 18, wherein the length of one or more sides of said square or rectangle cross-section is from about 0.6 mm to about 2.0 mm.

20. A histological specimen-substrate composition comprising:
(a) a planar or substantially planar substrate, and
(b) one or more histological specimens, each histological specimen comprising a pseudo-tissue sample or sub-portion thereof, said pseudo-tissue sample comprising an embedding medium and a clonal population of cells, wherein said clonal population of cells comprise fixed cells that have been collected into an aggregation and are embedded within said embedding medium; wherein said one or more histological specimens are in contact with at least one side of said substrate.

21. The histological specimen-substrate composition of claim 20, wherein said substrate is a microscope slide.

22. The histological specimen-substrate composition of claim 20, comprising from about 2 to about 1000 histological specimens.

23. The histological specimen-substrate composition of claim 20, comprising from about 10 to about 450 histological specimens.

24. The histological specimen-substrate composition of claim 20, comprising from about 50 to about 450 histological specimens.

25. The histological specimen-substrate composition of claim 20, comprising from about 100 to about 450 histological specimens.

26. The histological specimen-substrate composition of claim 20, comprising from about 200 to about 450 histological specimens.

27. The histological specimen-substrate composition of claim 22, wherein each or approximately each of said histological specimens comprise a distinct pseudo-tissue sample not represented by any other histological specimen located on said histological specimen-substrate composition.

28. The histological specimen-substrate composition of claim 22, wherein said histological specimens are arranged in an ordered two-dimensional array on said substrate.

29. A method for preparing a pseudo-tissue sample, said method comprising:
(a) obtaining a clonal population of cells,
(b) fixing the cells of said clonal population of cells,
(c) forming the fixed cells into an aggregation, and
(d) embedding said aggregation of cells in an embedding medium;
wherein said embedded aggregation of fixed cells comprises said pseudo-tissue sample.

30. The method of claim 29, wherein said clonal population of cells is comprised of mammalian cells.

31. The method of claim 29, wherein said clonal population of cells is comprised of hybrid cells.

32. The method of claim 31, wherein said hybrid cells are radiation hybrid cells.

33. The method of claim 29, wherein the cells of said clonal population of cells are fixed by contacting said cells with a solution comprising a fixative agent.

34. The method of claim 33, wherein said fixative agent is selected from the group comprising formaldehyde, formalin, glutaraldehyde, methanol, and ethanol.

35. The method of claim 34, wherein said fixative agent is formalin.

36. The method of claim 29, wherein said fixed cells are formed into an aggregation by centrifugation or filtration.

37. The method of claim 29, wherein said embedding medium comprises an ingredient selected from the group consisting of paraffin, gelatin, agar, wax, polyethylene glycol, polyvinyl alcohol, celloidin, nitrocellulose, methyl and butyl methacrylate resins, and epoxy resins.

38. The method of claim 37, wherein said embedding medium comprises paraffin.

39. A method for preparing a pseudo-tissue sample, said method comprising:
(a) obtaining two or more clonal populations of cells,
(b) fixing the cells of said two or more clonal populations of cells,
(c) forming the fixed cells into a single aggregation, and
(d) embedding said aggregation of cells in an embedding medium;
wherein said embedded aggregation of fixed cells comprises said pseudo-tissue sample.

40. A method for preparing a histological specimen, said method comprising:
(a) obtaining a pseudo-tissue sample, said pseudo-tissue sample comprising an embedding medium and a clonal population of cells, wherein said clonal population of cells comprise fixed cells that have been formed into an aggregation and are embedded within said embedding medium; and
(b) processing said pseudo-tissue sample so that said pseudo-tissue sample is suitable for microscopic analysis.

41. The method of claim 40, wherein said processing comprises removing a sub-portion from said pseudo-tissue sample.

42. The method of claim 41, wherein said sub-portion is a thin section removed from said pseudo-tissue sample.

43. The method of claim 42, wherein said thin section is removed from said pseudo-tissue sample with a microtome.

44. The method of claim 41, wherein said sub-portion is a core removed from said pseudo-tissue sample.

45. The method of claim 44, wherein said core is removed from said pseudo-tissue sample with a mechanical core punching device.

46. A method for preparing an array of histological specimens, wherein each histological specimen comprises a pseudo-tissue sample, said method comprising:
(a) obtaining two or more pseudo-tissue samples, wherein each pseudo-tissue sample comprises an embedding medium and a distinct clonal population of cells, wherein said clonal population of cells comprise fixed cells that have been collected into an aggregation and are embedded within said embedding medium;
(b) processing each pseudo-tissue sample so that each pseudo-tissue sample is suitable for microscopic analysis; and
(c) placing said processed pseudo-tissue samples on a planar or substantially planar substrate;
thereby producing an array of histological specimens.

47. The method of claim 46, wherein said processing comprises removing a sub-portion from said two or more pseudo-tissue samples.

48. The method of claim 47, wherein said sub-portion is a thin section of said two or more pseudo-tissue samples.

49. The method of claim 47, wherein said sub-portion is a core of said two or more pseudo-tissue samples.

50. The method of claim 46, wherein said substrate is a microscope slide.

51. A method for analyzing a tissue sample, said method comprising:
(a) obtaining one or more reference histological specimens, each reference histological specimen comprising a pseudo-tissue sample, said pseudo-tissue sample comprising an embedding medium and a clonal population of cells, wherein said clonal population of cells comprise fixed cells that have been collected into an aggregation and are embedded within said embedding medium;
(b) obtaining one or more candidate histological specimens, each candidate histological specimen comprising a tissue sample to be analyzed; and
(c) detecting one or more subcellular features common to said reference and candidate histological specimens.

52. The method of claim 51, wherein said pseudo-tissue is a pseudo-tissue sample that has been subjected to further processing so that said pseudo-tissue sample is suitable for microscopic analysis.

53. The method of claim 52, wherein said processing comprises removing a sub-portion from said pseudo-tissue sample.

54. The method of claim 53, wherein said sub-portion is a thin section of said pseudo-tissue sample.

55. The method of claim 53, wherein said sub-portion is a core of said pseudo-tissue sample.

56. The method of claim 51, wherein said tissue sample to be analyzed is obtained from a tumor.

57. The method of claim 51, wherein said fixed cells comprise fixed mammalian cells.

58. The method of claim 51, wherein said fixed cells comprise fixed hybrid cells.

59. The method of claim 58, wherein said fixed hybrid cells are fixed radiation hybrid cells.

60. The method of claim 51, wherein said one or more subcellular features that are detected are selected from the group consisting of cellular morphology, a nucleic acid, a polypeptide, a carbohydrate, and enzymatic activity.

61. The method of claim 51, wherein said one or more subcellular features are detected by a method comprising contacting said reference and candidate histological specimens with a reagent that facilitates the detection of said one or more subcellular features.

62. The method of claim 61, wherein said reagent that facilitates the detection of said one or more subcellular features comprises an antibody.

63. The method of claim 62, wherein said antibody is detectably labeled.

64. The method of claim 61, wherein said reagent that facilitates the detection of said one or more subcellular features comprises a nucleic acid probe.

65. The method of claim 64, wherein said nucleic acid probe is detectably labeled.

66. The method of claim 61, wherein said reagent that facilitates the detection of said one or more subcellular features comprises a compound that detects a carbohydrate.

67. The method of claim 61, wherein said reagent that facilitates the detection of said one or more subcellular features comprises a compound that is capable of detecting enzymatic activity.

68. A method for analyzing a plurality of tissue samples, said method comprising:
(a) obtaining one or more reference histological specimens, each reference histological specimen comprising a pseudo-tissue sample, said pseudo-tissue sample comprising an embedding medium and a clonal population of cells, wherein said clonal population of cells comprise fixed cells that have been collected into an aggregation and are embedded within said embedding medium;
(b) obtaining a plurality of candidate histological specimens, each candidate histological specimen comprising a distinct tissue sample to be analyzed; and
(c) detecting one or more subcellular features common to said reference and candidate histological specimens.

69. The method of claim 68, wherein said pseudo-tissue is a pseudo-tissue sample that has been subjected to further processing so that said pseudo-tissue sample is suitable for microscopic analysis.

70. The method of claim 69, wherein said processing comprises removing a sub-portion from said pseudo-tissue sample.

71. The method of claim 70, wherein said sub-portion is a thin section of said pseudo-tissue sample.

72. The method of claim 70, wherein said sub-portion is a core of said pseudo-tissue sample.

73. The method of claim 68, wherein said reference and candidate histological specimens are arranged in an ordered two-dimensional array that facilitates automated analysis.

74. The method of claim 68, wherein said one or more subcellular features are detected by a method comprising contacting said reference and candidate histological specimens with a reagent that facilitates the detection of said one or more subcellular features.

75. A method for identifying a tissue sample that possesses a pre-selected subcellular feature, said method comprising:
(a) obtaining one or more reference histological specimens, each reference histological specimen comprising a pseudo-tissue sample, said pseudo-tissue sample comprising an embedding medium and a clonal population of cells, wherein said clonal population of cells comprise fixed cells that have been collected into an aggregation and are embedded within said embedding medium, and wherein said clonal population of cells possess said pre-selected subcellular feature;
(b) obtaining a plurality of candidate histological specimens, each candidate histological specimen comprising a distinct tissue sample to be analyzed for the presence of said pre-selected subcellular feature; and
(c) detecting said pre-selected subcellular feature in said reference and candidate histological specimens.

76. The method of claim 75, wherein said pseudo-tissue is a pseudo-tissue sample that has been subjected to further processing so that said pseudo-tissue sample is suitable for microscopic analysis.

77. The method of claim 76, wherein said processing comprises removing a sub-portion from said pseudo-tissue sample.

78. The method of claim 77, wherein said sub-portion is a thin section of said pseudo-tissue sample.

79. The method of claim 77, wherein said sub-portion is a core of said pseudo-tissue sample.

80. The method of claim 75, wherein said pre-selected subcellular feature is detected by a method comprising contacting said reference and candidate histological specimens with a reagent that facilitates the detection of said pre-selected subcellular feature.

81. A kit for preparing a pseudo-tissue sample, said kit comprising:
(a) clonal population of cells;
(b) one or more solution comprising a fixative agent; and
(c) embedding medium;
wherein the kit components can be combined to form a pseudo-tissue sample,
wherein said pseudo-tissue sample comprises an embedding medium and a clonal population of cells, wherein said clonal population of cells comprise fixed cells that have been collected into an aggregation and are embedded within said embedding medium.

82. The kit of claim 81, further comprising one or more component selected from the group consisting of:
(a) one or more instrument for obtaining a thin section or core from a pseudo-tissue sample;
(b) one or more substrate upon which histological specimens may be placed for microscopic analysis;
(c) one or more reagent that facilitates the detection of one or more subcellular features;
(d) water;
(e) one or more buffer;
(f) one or more containers for storing or transporting kit components;
(g) computer software that facilitates the automated analysis of an array of histological specimens; and
(h) user instructions for preparing a pseudo-tissue sample or an array or pseudo-tissue samples.

83. The kit of claim 82, wherein said kit components can be combined to construct an array of pseudo-tissue samples, said array comprising two or more pseudo-tissue samples, wherein each pseudo-tissue sample is applied to a different discrete location on a substrate.

84. A kit for analyzing a tissue sample, said kit comprising:
(a) an array of reference histological specimens, each reference histological specimen comprising a pseudo-tissue sample;
(b) one or more solution comprising a fixative agent; and
(c) embedding medium;

85. The kit of claim 84, further comprising one or more component selected from the group consisting of:
(a) one or more instrument for obtaining a thin section or core from a pseudo-tissue sample;
(b) one or more substrate upon which histological specimens may be placed for microscopic analysis;
(c) one or more reagent that facilitates the detection of one or more subcellular features;
(d) water;
(e) one or more buffer;
(f) one or more containers for storing or transporting kit components;
(g) computer software that facilitates the automated analysis of an array of histological specimens; and
(h) user instructions for analyzing a tissue sample.

86. A composition comprising a plurality of pseudo-tissue samples, wherein each pseudo-tissue sample comprises an embedding medium and a clonal population of cells, wherein said clonal population of cells comprise fixed cells that have been collected into an aggregation and are embedded within said embedding medium.

87. A composition comprising 2 to 10 pseudo-tissue samples, wherein each pseudo-tissue sample comprises an embedding medium and a clonal population of cells, wherein said clonal population of cells comprise fixed cells that have been collected into an aggregation and are embedded within said embedding medium.

88. A composition comprising 2 to 50 pseudo-tissue samples, wherein each pseudo-tissue sample comprises an embedding medium and a clonal population of cells, wherein said clonal population of cells comprise fixed cells that have been collected into an aggregation and are embedded within said embedding medium.

89. A composition comprising 2 to 100 pseudo-tissue samples, wherein each pseudo-tissue sample comprises an embedding medium and a clonal population of cells, wherein said clonal population of cells comprise fixed cells that have been collected into an aggregation and are embedded within said embedding medium.
